SEQUENCE LISTING (16 sequences)

SEQ ID NO: 1 - protein, 29 aa, synthetic construct
RVLNLGPITRKGVQATVSRAFSSLGLLKV

SEQ ID NO: 2 - protein, 30 aa, synthetic construct
RVLNLGPITRKGGQAAMSRAAPSSLGLLQV

SEQ ID NO: 3 - protein, 30 aa, synthetic construct
RVLNLGPITRKNVQAVVSRAASSSLGFLKV

SEQ ID NO: 4 - protein, 30 aa, synthetic construct
RVLNLGPITRQGVQASVSKAASSNLGFLSI

SEQ ID NO: 5 - protein, 30 aa, synthetic construct
RVLNLGPITRQGVQASVSKAASSNLRLLSI

SEQ ID NO: 6 - RNA, 21 nt, synthetic construct
  source 1..21: mol_type = other RNA
gtctacctgcactgtgaagtc

SEQ ID NO: 7 - RNA, 20 nt, synthetic construct
  source 1..20: mol_type = other RNA
agactttcaggagccctttc

SEQ ID NO: 8 - RNA, 22 nt, synthetic construct
  source 1..22: mol_type = other RNA
gtgactctacgagtgaacagtg

SEQ ID NO: 9 - RNA, 20 nt, synthetic construct
  source 1..20: mol_type = other RNA
cagatgctcaggagcccttg

SEQ ID NO: 10 - RNA, 20 nt, synthetic construct
  source 1..20: mol_type = other RNA
aagacgagaaacatgagaag

SEQ ID NO: 11 - RNA, 20 nt, synthetic construct
  source 1..20: mol_type = other RNA
tggacacctttgtatgaaac

SEQ ID NO: 12 - RNA, 20 nt, synthetic construct
  source 1..20: mol_type = other RNA
tggacactgaggcctggaca

SEQ ID NO: 13 - RNA, 22 nt, synthetic construct
  source 1..22: mol_type = other RNA
gtacagaaagaacctaaactta

SEQ ID NO: 14 - RNA, 21 nt, synthetic construct
  source 1..21: mol_type = other RNA
tggacacctttggaggaaaac

SEQ ID NO: 15 - RNA, 20 nt, synthetic construct
  source 1..20: mol_type = other RNA
cttgagactgtggcctggac

SEQ ID NO: 16 - RNA, 22 nt, synthetic construct
  source 1..22: mol_type = other RNA
ctgcagaaaggacctgaactta

(19) United States
(12) Patent Application Publication
(10) Pub. No.: US 2025/0145993 A1
(43) Pub. Date: May 8, 2025

(54) ENHANCED EXPRESSION OF ALTERNATIVE SPLICED UROMODULIN FOR THERAPEUTICUSE

(71) Applicant: The Trustees of Indiana University, Bloomington, IN (US)

(72) Inventors: Tarek Maurice Ashkar, Carmel, IN (US); Azuma Nanamatsu, Indianapolis, IN (US)

(73) Assignee: The Trustees of Indiana University, Bloomington, IN (US)

(21) Appl. No.: 18/934,880

(22) Filed: Nov. 1, 2024

Related U.S. Application Data
(60) Provisional application No. 63/595,436, filed on Nov. 2, 2023.

(51) Int. Cl. C12N 15/113; A61P 13/12

(52) U.S. Cl. CPC C12N 15/113; A61P 13/12; C12N 2310/11; C12N 2320/33

(57) ABSTRACT

A method of enhancing the presence of alternatively-spliced UMOD protein by inducing exon-skipping of a AS-UMOD m-RNA to protect TAL cells of kidneys in a patient suffering from acute kidney injury or chronic kidney disease.

### CROSS-REFERENCE TO RELATED APPLICATIONS

This application claims the benefit under 35 U.S.C. § 119(e) of U.S. Provisional Application No. 63/595,436 entitled “ENHANCED EXPRESSION OF ALTERNATIVE SPLICED UROMODULIN FOR THERAPEUTIC USE, filed on Nov. 2, 2023, of which is incorporated by reference in its entirety.

### STATEMENT OF GOVERNMENTAL RIGHTS

This invention was made with government support under BX003935 merit award by the Veterans Administration. The government has certain rights in the invention.

### REFERENCE TO SEQUENCE LISTING

The instant application contains a Sequence Listing XML which has been submitted electronically and is hereby incorporated by reference in its entirety. Said Sequence Listing XML copy, created on Oct. 28, 2024 is named “IU202403502USST26.xml” and is 15,173 bytes in size.

### FIELD OF THE INVENTION

The present disclosure relates generally to the fields of medicine and kidney injury and disease. In particular, it relates to enhanced expression of alternative spliced uromodulin for protection and treatment of kidney cells.

### BACKGROUND

Acute kidney injury (AKI) denotes a sudden and often reversible reduction in the kidney function. AKI can lead to the accumulation of water, sodium, and other metabolic products. It can also result in several electrolyte disturbances. It is a very common condition, especially among hospitalized patients. It can be seen in up to 7% of hospital admissions and 30% of ICU admissions.

Although the injury and repair of proximal tubule (PT) cells have been intensively studied as a crucial factor in acute kidney injury (AKI) and as a factor in progression to chronic kidney disease (CKD), the impact of damaged thick ascending limb (TAL) cells on kidney injury has been underrecognized up until recently. Both thick ascending limb (TAL) and proximal tubule (PT) cells are enriched in mitochondria for ATP-driven ion transport in the kidney. The TAL composes approximately 20% of all cells in the kidney. This segment reabsorbs 25% of the filtered Na+. In a transgenic mouse model, targeted injury to TAL cells was sufficient to cause severe AKI (Srichai, M. B., et al., Apoptosis of the thick ascending limb results in acute kidney injury. J Am Soc Nephrol, 2008. 19(8): p. 1538-46). Recent single-cell analysis in human AKI showed that TAL cells express injury markers, suggesting that TAL cells are also injured (Hinze, C., et al., Single-cell transcriptomics reveals common epithelial response patterns in human acute kidney injury. Genome Med, 2022. 14(1): p. 103). The latest multimodal single-cell and spatial analysis on healthy and diseased human kidneys from the Kidney Precision Medicine Project identified adaptive (successful or maladaptive repair) and degenerative (damaged or stressed) TAL cell subtypes. Importantly, this study demonstrated the unique and independent association of injured TAL signature with kidney disease progression (Lake, B. B., et al., An atlas of healthy and injured cell states and niches in the human kidney. Nature, 2023. 619(7970): p. 585-594). Furthermore, molecules produced by TAL cells such as uromodulin (UMOD) and epidermal growth factor remain the most reliable in predicting adaptive repair and kidney recovery in experimental models and in large human cohorts such as the ASSESS-AKI study (Puthumana, J., et al., Biomarkers of inflammation and repair in kidney disease progression. J Clin Invest, 2021. 131(3); Wen, Y., et al., Longitudinal biomarkers and kidney disease progression after acute kidney injury. JCI Insight, 2023. 8(9). These findings suggest that injury of TAL cells is likely very detrimental for the severity of injury and prospects of adaptive repair. Therefore, methods for enhancing TAL protection, as described herein, are important for resistance to kidney injury and better recovery and outcomes in patients.

### SUMMARY OF THE INVENTION

A first aspect of the invention includes methods for enhancing endogenous alternatively spliced uromodulin mRNA expression in a subject.

A second aspect of the invention includes methods for treating kidney injury in a subject by increasing the presence of alternatively spliced uromodulin protein in the subject.

A first embodiment being a method of inducing exon-skipping of a AS-UMOD m-RNA in a TAL cell, the method comprising delivering to the cell a splice-switching antisense oligonucleotides (SSO) comprising a sequence selected from the group consisting of SEQ ID NOs: 14, 15, and 16, wherein the antisense oligonucleotide is up to 30 nucleotides in length and is capable of inducing exon-skipping of the UMOD pre-mRNA.

A second embodiment being a method of inducing exon-skipping of a AS-UMOD m-RNA in a TAL cell, the method comprising delivering to the cell a splice-switching antisense oligonucleotides (SSO), wherein the SSO sequence comprises SEQ ID NO: 14.

A third embodiment being a method of inducing exon-skipping of a AS-UMOD m-RNA in a TAL cell, the method comprising delivering to the cell a splice-switching antisense oligonucleotides (SSO), wherein the SSO sequence comprises SEQ ID NO: 15.

A fourth embodiment being a method of inducing exon-skipping of a AS-UMOD m-RNA in a TAL cell, the method comprising delivering to the cell a splice-switching antisense oligonucleotides (SSO), wherein the SSO sequences comprises SEQ ID NO: 16.

A fifth embodiment being a method for enhancing endogenous alternatively spliced uromodulin (AS-UMOD) expression in a subject by administering splice-switching antisense oligonucleotides (SSO) comprising a sequence selected from the group consisting of SEQ ID NOs: 14, 15, and 16 to a subject.

A sixth embodiment being a method for enhancing endogenous alternatively spliced uromodulin (AS-UMOD) expression in a subject by administering splice-switching antisense oligonucleotides (SSO) comprising a sequence selected from the group consisting of SEQ ID NOs: 14, 15, and 16 to a subject., wherein the enhanced endogenous AS-UMOD mRNA expression occurs in the kidney of the subject.

A seventh embodiment being a method for enhancing endogenous alternatively spliced uromodulin (AS-UMOD) expression in a subject by administering splice-switching antisense oligonucleotides (SSO) comprising a sequence selected from the group consisting of SEQ ID NOs: 14, 15, and 16 to a subject. wherein the enhanced endogenous AS-UMOD mRNA expression occurs in the TAL cells of the subject

An eighth embodiment being a method for enhancing endogenous alternatively spliced uromodulin (AS-UMOD) expression in a mammalian subject by administering splice-switching antisense oligonucleotides (SSO) comprising a sequence selected from the group consisting of SEQ ID NOs: 14, 15, and 16 to a mammalian subject.

A ninth embodiment being a method for enhancing endogenous alternatively spliced uromodulin (AS-UMOD) expression in a human subject by administering splice-switching antisense oligonucleotides (SSO) comprising a sequence selected from the group consisting of SEQ ID NOs: 14, 15, and 16 to a human subject.

A tenth embodiment being a method for enhancing endogenous alternatively spliced uromodulin (AS-UMOD) expression in a subject by administering splice-switching antisense oligonucleotides (SSO) comprising a sequence selected from the group consisting of SEQ ID NOs: 14, 15, and 16 to a subject, wherein the AS-UMOD expressed lacks exon 10.

An eleventh embodiment being a method for treating acute kidney injury in a subject by enhancing endogenous alternatively spliced uromodulin (AS-UMOD) expression in a subject by administering splice-switching antisense oligonucleotides (SSO) comprising a sequence selected from the group consisting of SEQ ID NOs: 14, 15, and 16 to a subject.

A twelfth embodiment being a spice-switching antisense oligonucleotide (SSO) consisting of a sequence that has at least 90% identical over the entire length of SEQ ID NO: 14, 15 or 16.

A thirteenth embodiment being a method of use of a composition containing a spice-switching antisense oligonucleotide (SSO) consisting of a sequence that has at least 90% identical over the entire length of SEQ ID NO: 14, 15 or 16 in a subject suffering kidney injury.

A fourteenth embodiment being a method of treating acute kidney injury in a subject wherein the splice-switching antisense oligonucleotides (SSO) is administered to the subject less than 72 hours following acute kidney injury in the subject.

A fifteenth embodiment being a method of enhancing endogenous alternatively spliced uromodulin mRNA expression in a subject where the splice-switching antisense oligonucleotides (SSO) is administered to the subject prior to surgery on the kidney of the subject.

A sixteenth embodiment being a method of enhancing endogenous alternatively spliced uromodulin mRNA expression in a subject where the splice-switching antisense oligonucleotides (SSO) is administered to the subject less than 72 hours following acute kidney injury in the subject.

A fourteenth embodiment being a pharmaceutical composition comprising a pharmaceutically acceptable carrier and two or more antisense oligonucleotides complementary to AS-UMOD, wherein at least one of said antisense oligonucleotides thereof comprises a nucleic acid sequence as set forth in any one of SEQ ID Nos: 14, 15 or 16.

### BRIEF DESCRIPTION OF THE FIGURES

The accompanying drawings are included to provide a further understanding of the disclosure and are incorporated in and constitute a part of this specification, illustrate embodiments, and together with the description serve to explain the principles of the disclosure.

FIG. 1A is a graphical representation of the structure and domains of the UMOD protein. The structure and domains of the UMOD protein are designated as follows: the Roman numerals 1 through 4 indicate the four EGF-like domains; D8C indicate 8-cysteine domain; ZP indicates zona pellucida; and EHP indicates external hydrophobic patch.

FIG. 1B is a comparison of the C-terminal exons and sequences of UMOD for human and several other mammalian species. GPI indicates glycosylphosphatidylinositol.

FIG. 2A is a representative Sashimi Plot of long-read RNA sequence of UMOD gene in human kidney.

FIG. 2B is a representative Sashimi Plot of long-read RNA sequence of UMOD gene in mouse kidney.

FIG. 2C is an image ofRT-PCR products for exon 10 skipping or exon 10 retaining UMOD in human kidney.

FIG. 2D is an image ofRT-PCR products for exon 10 skipping or exon 10 retaining UMOD in mouse kidney.

FIG. 2E is sanger sequencing of exon 10 skipping UMOD in human kidney.

FIG. 2F is sanger sequencing of exon 10 skipping UMOD in mouse kidney.

FIG. 2G is a graphical illustration of canonical UMOD (C-UMOD) and Alternatively-spliced UMOD (AS-UMOD).

FIG. 3A is a bar graph showing relative mRNA expression of AS-UMOD in IRI mice. Wild-type male mice were subjected to sham or bilateral mild IRI (22 min clamp-time) or severe IRI surgery (30 min clamp-time). Kidney mRNA were isolated 24 hours after the surgery. Mild IRI up-regulated AS-UMOD expression, whereas severe IRI did not induce AS-UMOD. *p<0.05, ns: not significant.

FIG. 3B is a bar graph showing relative mRNA expression of C-UMOD in IRI mice. Wild-type male mice were subjected to sham or bilateral mild IRI (22 min clamp-time) or severe IRI surgery (30 min clamp-time). Kidney mRNA were isolated 24 hours after the surgery. Mild IRI up-regulated AS-UMOD expression, whereas severe IRI did not induce AS-UMOD. *p<0.05, ns: not significant.

FIG. 4A is a tSNE plot (with G means clustering) showing various cell types in mouse kidney using 32 markers for epithelial, endothelial, immune and injury markers. TAL cells were further clustered based on injury markers.

FIG. 4B is an image of spatial analysis using Co-detection by Indexing (CODEX) multi-fluorescence imaging CODEX to delineate TAL cell subpopulations. Large scale image of an injured kidney (22 minute clamp) showing various cell type distribution.

FIG. 4C is an image of spatial analysis using Co-detection by Indexing (CODEX) multi-fluorescence imaging CODEX to delineate TAL cell subpopulations. Large scale image of an injured kidney (22 minute clamp) showing 2 TAL subtypes.

FIG. 4D is an image of spatial analysis using Co-detection by Indexing (CODEX) multi-fluorescence imaging CODEX to delineate TAL cell subpopulations. Injured TALs (cluster 1, p-C-JUN high) map predominantly to the medulla.

FIG. 4E is a graphical illustration of spatial analysis using Co-detection by Indexing (CODEX) multi-fluorescence imaging CODEX to delineate TAL cell subpopulations. The protein expression profile of the 5 TAL subclusters.

FIG. 4F is an illustration of spatial analysis using Co-detection by Indexing (CODEX) multi-fluorescence imaging CODEX to delineate TAL cell subpopulations. Rabbit polyclonal AS-UMOD specifically reacts with Exon 10 skipping peptide. Control Antibody's epitope is supped to be within exon 9 and reacts with both peptides.

FIG. 5A is a collection of images of immunofluorescence of UMOD in MDCK cells tranduced with C-UMOD or AS-UMOD. Bars=10 μm.

FIG. 5B is a collection of images of immunofluorescence of UMOD and mitochondria (Mitotracker) in MDCK cells tranduced with C-UMOD or AS-UMOD. Co-localization was quantified using the Manders' colocalization coefficient. Co-occurrence of the two signals was determined using tM1 and tM2. tM1 is considered as a readout of UMOD localizing mitochondria.

FIG. 5C is a bar graph of the results of FIG. 5B showing the co-localization using the Manders' colocalization coefficient.

FIG. 5D is an image of Western blotting of UMOD in MDCK cells after subcellular fractionation. AS-UMOD was mainly localized at mitochondria (highlighted in red). Isolation of mitochondrial fraction was validated by mitochondrial marker (VDAC1). *p<0.05.

FIG. 6A. Bar graph depicting seahorse XFp Cell Mito stress test in MDCK cells expressing C-UMOD or AS-UMOD.

FIG. 6B is a bar graph depicting seahorse XFp Cell Mito stress test in MDCK cells expressing C-UMOD or AS-UMOD.

FIG. 6C is a bar graph depicting the mitochondrial area/cell area of cells expressing C-UMOD or AS-UMOD.

FIG. 6D is a representative image of transmission electron microscopy in MDCK cells expressing C-UMOD. Bar=1 μm. Mitochondrial mass normalized to cell was quantitated (right). *p<0.05, ns: not significant.

FIG. 6E is a representative image of transmission electron microscopy in MDCK cells expressing AS-UMOD. Bar=1 μm. Mitochondrial mass normalized to cell was quantitated (right). *p<0.05, ns: not significant.

FIG. 7A is a bar graph depicting lactate dehydrogenase (LDH) reslease in MDCK cells expressing AS-UMOD. MDCK cells expressing C-UMOD or AS-UMOD were cultured in normoxia or hypoxia condition for 6 h. Media was corrected and LDH concentration was measured. 48 hr after transfection, cells were cultured in normoxia or hypoxia condition for 6 h. Media was corrected and LDH concentration was measured. **p<0.01, ***p<0.001, ****p<0.0001.

FIG. 7B is a bar graph depicting lactate dehydrogenase (LDH) release in MKTAL cells transfected with SSOs. MKTAL cells were transfected with 30 nM non-targeted (negative control) or UMOD-exon 10 targeted SSOs using lipofectamine 2000. 48 hr after transfection, cells were cultured in normoxia or hypoxia condition for 6 h. Media was corrected and LDH concentration was measured. **p<0.01, ***p<0.001, ****p<0.0001.

FIG. 8 is a bar graph depicting relative AS-UMOD/C-UMOD mRNA expression of in the murine kidney transfected with SSO. Wild-type male mice were administered with PBS (control) or 25 mg/kg SSO by retro-orbital injection and harvested 48 hr after injection. p<0.05.

FIG. 9A is a schematic of an experimental design. AS-UMOD induction protects TAL cells and ameliorates severe IRI. Wild-type mice underwent severe IRI and SSO treatment (25 mg/kg) and were harvested 72 hours after IRI.

FIG. 9B is a bar graph depicting relative mRNA expression of AS-Umod and C-Umod normalized to Gapdh of the experiment of FIG. 9A.

FIG. 9C is a collection of images of immunofluorescence of murine kidneys from the experiment of FIG. 9A. The white arrows indicate AS-UMOD which is induced in the cytosol of TAL cells after Umod SSO treatment. Scale Bar=50 μm.

FIG. 9D is a graphical illustration of serum urea concentration from the experiment of FIG. 9A.

FIG. 9E is an images of PAS-stained kidney sections from the experiment of FIG. 9A.

FIG. 9F is a graph of the quantification of injury of the results of the experiment in FIG. 9A. Scale Bar=500 μm.

FIG. 9G is a graphical illustration of the relative mRNA expression of injury-related genes normalized to Gapdh of the experiment shown in FIG. 9A. Values are presented as mean±SEM. *p<0.05, ****p<0.0001.

### DETAILED DESCRIPTION

We identified an alternatively-spliced UMOD (AS-UMOD) that skips exon 10 in human and mouse kidneys. AS-UMOD protects TAL cells through intracellular targeting that enhances mitochondrial metabolism. As described herein, we have developed slice-switching antisense oligonucleotides (SSOs) that can be used to modulate AS-UMOD expression in TAL cells. The SSOs induce exon 10 skipping during transcription of the UMOD gene, generating a splice variant of UMOD which lacks exon 10. This allows more AS-UMOD to be present in the to protect the TAL cells.

Uromodulin (UMOD, also known as Tamm-Horsfall protein) is a kidney-specific protein expressed specifically in cells of thick ascending limbs (TAL). UMOD enters the secretory pathway and is released bidirectionally into the urine and circulation. Genome-Wide Association Studies revealed that single nucleotide polymorphisms in the UMOD regulatory region are highly associated with acute kidney injury (AKI) and chronic kidney disease (CKD), highlighting the relevance of UMOD in kidney injury (Kottgen, A., et al., Multiple loci associated with indices of renal function and chronic kidney disease. Nat Genet, 2009. 41(6): p. 712-7; Kottgen, A., et al., New loci associated with kidney function and chronic kidney disease. Nat Genet, 2010. 42(5): p. 376-84; Akwo, E. A., et al., Phenome-Wide Association Study of UMOD Gene Variants and Differential Associations With Clinical Outcomes Across Populations in the Million Veteran Program a Multiethnic Biobank. Kidney Int Rep, 2022. 7(8): p. 1802-1818; El-Achkar, T. M., et al., Tamm-Horsfall protein translocates to the basolateral domain of thick ascending limbs, interstitium, and circulation during recovery from acute kidney injury. Am J Physiol Renal Physiol, 2013. 304(8): p. F1066-75). However, the mechanisms of such modulatory role(s) of UMOD had not been fully elucidated. Recent studies demonstrated protective functions of UMOD against kidney injury, and clinical observational studies have linked higher levels of secreted UMOD to better renal outcomes. We have previously demonstrated in experimental models that UMOD deficiency aggravates AKI and that UMOD is essential in the adaptive repair and recovery after kidney injury. Specifically, we have shown that basolaterally released UMOD terminates inflammatory signalling in proximal tubules (PT) (El-Achkar, T. M., et al., Tamm-Horsfall protein translocates to the basolateral domain of thick ascending limbs, interstitium, and circulation during recovery from acute kidney injury. Am J Physiol Renal Physiol, 2013. 304(8): p. F1066-75; El-Achkar, T. M., et al., Tamm-Horsfall protein-deficient thick ascending limbs promote injury to neighboring S3 segments in an MIP-2-dependent mechanism. Am J Physiol Renal Physiol, 2011. 300(4): p. F999-F1007) and regulates renal and systemic oxidative stress (LaFavers, K. A., et al., Circulating uromodulin inhibits systemic oxidative stress by inactivating the TRPM2 channel. Sci Transl Med, 2019. 11(512)). The importance of UMOD in adaptive repair and recovery is also supported by work from other groups, showing that maladaptive repair and progression of injury are associated with decreased expression of UMOD (Puthumana, J., et al., Biomarkers of inflammation and repair in kidney disease progression. J Clin Invest, 2021. 131(3); Wen, Y., et al., Longitudinal biomarkers and kidney disease progression after acute kidney injury. JCI Insight, 2023. 8(9).

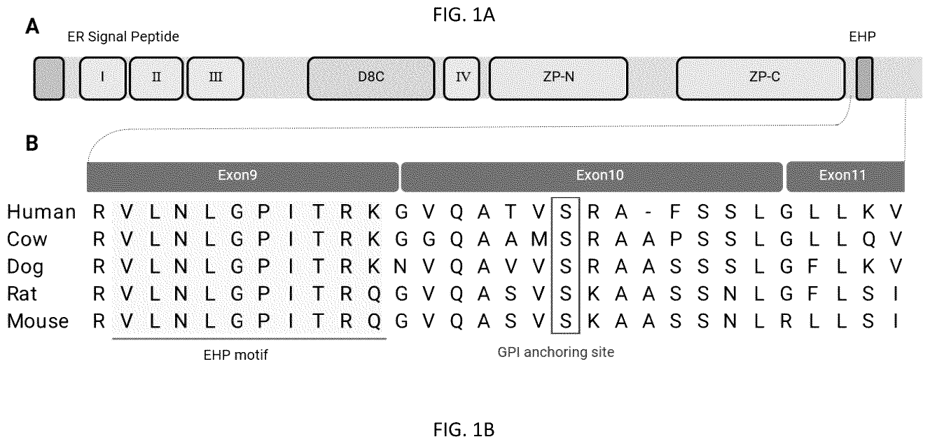

UMOD is a glycosylphosphatidylinositol (GPI)-anchored cell membrane protein and is secreted into urine and circulation. As illustrated in FIG. 1A, UMOD is synthesized as a precursor of 640 amino acids and is composed of 11 exons, of which exons 2 through 11 are the protein-coding region. At the N-terminus, UMOD contains endoplasmic reticulum (ER) signal peptides that guide the protein into the ER membrane and subsequently the secretory pathway. The C-terminus is important for intracellular targeting and extracellular secretion (See Micanovic, R., et al., The kidney releases a nonpolymerizing form of uromodulin in the urine and circulation that retains the external hydrophobic patch domain. Am J Physiol Renal Physiol, 2022. 322(4): p. F403-F418). A comparison of the C-terminal exons and sequence of UMOD in human (SEQ ID NO:1), cow (SEQ ID NO:2), dog (SEQ ID NO:3), Rat (SEQ ID NO:4), and mouse (SEQ ID NO:5) (See FIG. 1B) demonstrates that the sequence around the C-terminus is highly conserved between species. Specifically, exon 9 contains the external hydrophobic patch (EHP) motif, which inhibits intracellular polymerization and aggregation (Schaeffer, C., et al., Analysis of uromodulin polymerization provides new insights into the mechanisms regulating ZP domain-mediated protein assembly. Mol Biol Cell, 2009. 20(2): p. 589-99). Exon 10 contains a GPI-anchoring site, making this exon important for membrane sorting. However, prior to the present description, splice variants around the C-terminus have not been previously described.

TABLE 1
UMOD Sequence in Various Organisms

| SEQ | | |
|---|---|---|
| ID NO | Name | Sequence |
| 1 | Human UMOD | RVLNLGPITRKGVQATVSRA-FSSLGLLKV |
| 2 | Cow UMOD | RVLNLGPITRKGGQAAMSRAAPSSLGLLQV |
| 3 | Dog UMOD | RVLNLGPITRKNVQAVVSRAASSSLGFLKV |
| 4 | Rat UMOD | RVLNLGPITRQGVQASVSKAASSNLGFLSI |
| 5 | Mouse UMOD | RVLNLGPITRQGVQASVSKAASSNLRLLSI |

We have identified splice variants around the C-terminus. Most preferably, we have identified alternatively-spliced UMOD (AS-UMOD) which lacks the C-terminal exon 10 in both mouse and human kidneys. Our preliminary data supports the relevance of this variant in injury. First, AS-UMOD was specifically targeted to the mitochondria and was induced after AKI. Second, AS-UMOD interacted with glutamate and ADP/ATP carriers in the inner membrane of mitochondria and improved mitochondrial function. Third, enhanced expression of AS-UMOD improved the viability of TAL cells under hypoxia conditions. Notably, the localization and regulatory mechanism of AS-UMOD are contrasted with exon 10 retaining canonical UMOD (C-UMOD); C-UMOD was targeted to the secretory pathway and was decreased after AKI. These data suggest that the induction of AS-UMOD and its mitochondrial targeting are part of the protective mechanism for TAL cells in kidney injury.

Induction of AS-UMOD leads to a potential novel therapeutic strategy and a better understanding of spatial metabolism in AKI. We generated splice-switching antisense oligonucleotides (SSOs) that can enhance AS-UMOD expression in vitro and in vivo. Utilizing the protective role of AS-UMOD, we have developed a novel therapeutic approach to protecting and repairing TAL cells by enhancing AS-UMOD expression using SSOs. Through administration of SSOs to a subject diagnosed with AKI, the endogenous levels of AS-UMOD are enhanced and the severity of AKI in the subject may be mitigated.

For purpose or this disclosure, the term “exon skipping” is herein defined as inducing, producing or increasing production within a cell of a mature mRNA that does not contain a particular exon that would be present in the mature mRNA without exon skipping. Exon skipping is achieved by providing a cell expressing the pre-mRNA of said mature mRNA with a molecule capable of interfering with sequences. Examples of exon skipping molecules may include, molecules capable of interfering with the (cryptic) splice donor or (cryptic) splice acceptor sequence required for allowing the enzymatic process of splicing, or with a molecule that is capable of interfering with an exon inclusion signal required for recognition of a stretch of nucleotides as an exon to be included in the mature mRNA. The term pre-mRNA refers to a non-processed or partly processed precursor mRNA that is synthesized from a DNA template of a cell by transcription, such as in the nucleus.

The term “antisense oligonucleotide” is understood to refer to a nucleotide sequence which is substantially complementary to a target nucleotide sequence in a pre-mRNA molecule, hnRNA (heterogenous nuclear RNA) or mRNA molecule. The degree of complementarity (or substantial complementarity) of the antisense sequence is preferably such that a molecule comprising the antisense sequence can form a stable hybrid with the target nucleotide sequence in the RNA molecule under physiological conditions.

The terms “SSO”, “slice-switching antisense oligonucleotides,” “antisense oligonucleotide” and “oligonucleotide” are used interchangeably herein and are understood to refer to an oligonucleotide comprising an antisense sequence.

As used herein, the term “AKI” encompasses the criteria for diagnosis and/or classification currently utilized by those of skill in the art. In humans, these criteria may include, but are not limited to, the presence of any of the following: Increase in serum creatinine by 0.3 mg/dL or more (26.5 micromoles/L or more) within 48 hours; Increase in serum creatinine to 1.5 times or more baseline within the prior seven days; and/or Urine volume less than 0.5 mL/kg/h for at least 6 hours.

The methods disclosed herein are also applicable to the treatment of chronic kidney disease models (e.g., diabetic nephropathy) with UMOD deficiency where AS-UMOD induction could even be further impaired.

SSOs of the present invention may be administered to a subject in need thereof through any route of administration known to those in the art for delivering antisense molecules and oligonucleotides. It is possible that the effect size of AS-UMOD induction may need to be further augmented. In those cases, SSO administration may be enhanced using in vivo-jetPEI (Polyplus), a lipid-based transfection reagent developed to deliver DNA and RNA in vivo, which is efficient for delivery to the kidney (Zhu, Q., et al., Silencing of HIF prolyl-hydroxylase 2 gene in the renal medulla attenuates salt-sensitive hypertension in Dahl S rats. Am J Hypertens, 2014. 27(1): p. 107-13; Liu, Y., et al., Renal lysophospholipase Al contributes to Enterococcus faecalis-induced hypertension by enhancing sodium reabsorption. iScience, 2022. 25(12): p. 105403). Preferably, the SSO is administered through an intravenous route; however, administration through a subcutaneous or intraperitoneal are also contemplated herein and may have the benefit of a slower release and mitigate a first pass clearance by the liver.

Preliminary observations of administration of SSOs of the present invention have shown no evidence of toxicity in small mammal models, such as mice. Dosages of about 25 mg/kg in mice have shown therapeutic effect. The lack of toxicity allows a wide dosage window for the SSOs in mammals. SSOs of the present invention may be administered to human at therapeutic levels, including about 2 mg/kg, about 4 mg/kg, about 6 mg/kg, about 8 mg/kg, about 10 mg/kg, about 12 mg/kg, about 14 mg/kg, about 16 mg/kg, about 18 mg/kg and about 20 mg/kg.

Splice-switching antisense oligonucleotides (SSO) may be administered prior to potential kidney injury, such as prior to surgery on the kidney, in order to decrease the potential of acute kidney injury, as well as increase protection of the kidney and enhance recovery of any injury to the kidney. SSOs may also be administered immediately or within hours after injury to the kidney, such as after surgery on the kidney or after traumatic impact upon the kidney. Preferably, SSOs are administered within 72 hours of the occurrence of acute injury to the kidney. SSOs of the present invention may also be administered within hours or days of detection or diagnosis of AKI in the patient.

### EXAMPLES

### Example 1: Identification of AS-UMOD

We identified an alternatively-spliced UMOD that skips exon 10 in human and mouse kidneys. This variant of UMOD is referred to herein as AS-UMOD (alternatively-spliced UMOD). AS-UMOD is induced by murine kidney ischemia-reperfusion injury model and protects TAL cells through intracellular targeting that enhances mitochondrial metabolism.

Conventionally, short-lead RNA sequencing has been used to evaluate splice variants; however, short-reads might not span entire exons or introns, making it difficult to decipher the exact splicing pattern and overall view. We utilized long-read RNA sequencing technologies (Hu, Y., et al., LIQA: long-read isoform quantification and analysis. Genome Biol, 2021. 22(1): p. 182) to explore splicing variants of UMOD and detected alternatively-spliced UMOD (AS-UMOD), an isoform that has not been previously investigated.

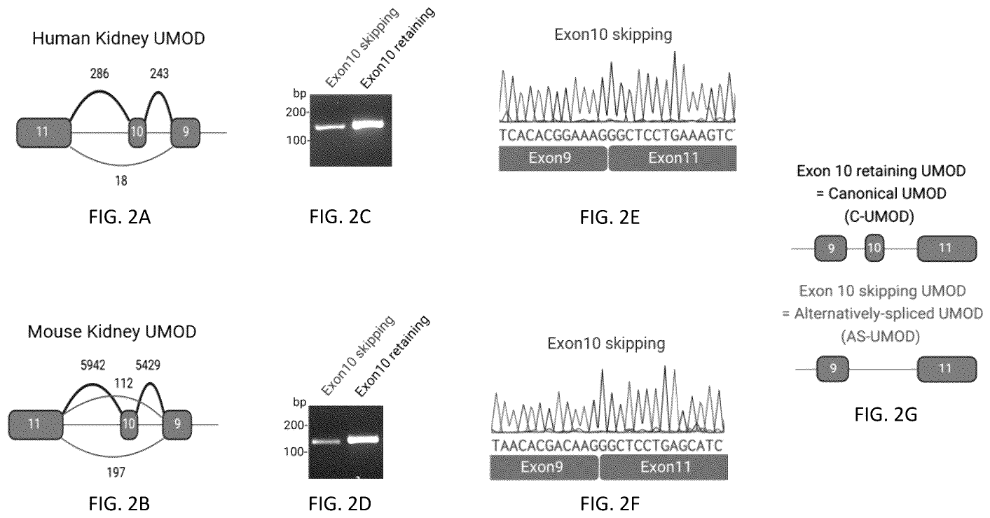

We expanded our understanding of UMOD biology by demonstrating that this protein, which is usually targeted towards secretory pathway, can be shifted intracellularly towards the mitochondria by alternative splicing at the C terminus. To identify the splice variants in the C-terminal region of UMOD, we performed long-read RNA sequencing, which revealed exon 10 skipping UMOD both in human and in mouse kidneys (FIG. 2A, 2B). The existence of exon 10 skipping UMOD mRNA was confirmed by RT-PCR followed by Sanger sequencing in human (FIG. 2C, 2E) and in mouse (FIG. 2D, 2F). Since exon 10 contains the GPI-anchoring site (FIG. 1B), the absence of exon 10 is likely to change the subcellular localization of UMOD and exert diverse effects. Furthermore, the presence of this splice variant in both human and mouse kidneys suggests an important function. Indeed, our data support a key protective function of this variant in kidney injury (discussed below). We defined exon 10 retaining UMOD as canonical UMOD (C-UMOD), and exon 10 skipping UMOD as alternatively-spliced UMOD (AS-UMOD) (FIG. 2G).

### Example 2: AS-UMOD as Indicator of Kidney Injury

Based on our preliminary data, AS-UMOD was induced during AKI and was targeted intracellularly towards mitochondria. Notably, the regulation and localization of AS-UMOD are contrasted with C-UMOD; the latter is directed towards the secretory pathway and is downregulated after early AKI.

We utilized a murine renal ischemia reperfusion injury (IRI) model which uses bilateral renal pedicle clamping to induce kidney injury in the mice. The IRI technique is described in El-Achkar, T. M., et al., Tamm-Horsfall protein-deficient thick ascending limbs promote injury to neighboring S3 segments in an MIP-2-dependent mechanism. Am J Physiol Renal Physiol, 2011. 300(4): p. F999-F1007 and El-Achkar, T. M., et al., Tamm-Horsfall protein protects the kidney from ischemic injury by decreasing inflammation and altering TLR4 expression. Am J Physiol Renal Physiol, 2008. 295(2): p. F534-44).

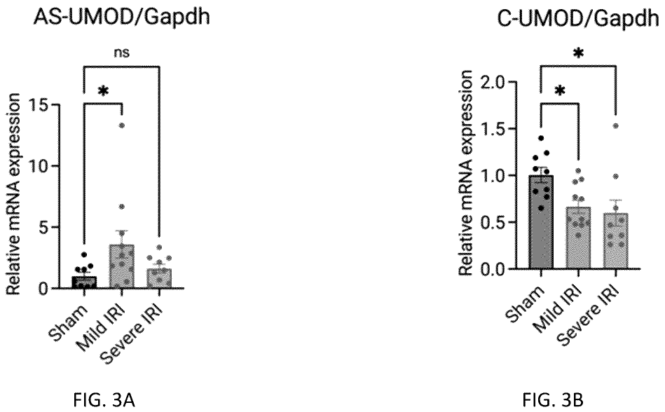

We have found that IRI induces AS-UMOD expression following a dynamic time course and localization that is linked with TAL exposure to injury; the AS-UMOD response is diminished with severe injury. Our preliminary data showed that AS-UMOD mRNA expression was up-regulated 24 hours after mild IRI (22 min clamp-time) (FIG. 3A). Importantly, C-UMOD mRNA was down-regulated at this time point, consistent with previous reports from our lab (FIG. 3B) (See for example El-Achkar, T. M., et al., Tamm-Horsfall protein translocates to the basolateral domain of thick ascending limbs, interstitium, and circulation during recovery from acute kidney injury. Am J Physiol Renal Physiol, 2013. 304(8): p. F1066-75). These results suggest that kidney injury induces a switch from generating C-UMOD towards AS-UMOD. Furthermore, severe IRI (30 min clamp-time) failed to up-regulate AS-UMOD (FIG. 3A). Losing this proposed protective mechanism during severe kidney injury could be detrimental to TAL cell integrity, which would negatively impact the course of injury and repair. Indeed, we have previously shown that severe IRI causes significant injury to TAL cells (histologically defined) (El-Achkar, T. M., et al., Tamm-Horsfall protein protects the kidney from ischemic injury by decreasing inflammation and altering TLR4 expression. Am J Physiol Renal Physiol, 2008. 295(2): p. F534-44).

We developed qRT-PCR primers that specifically detect AS-UMOD in humans and in mice. The forward primer is designed to bind to UMOD exon 8, and the reverse primer is designed to bind to the junction between exon 9 and 11. The Reverse primer is designed so that only 4-5 bases of the 3′ side are exon 9 sequences and not exon 10 in the retained mRNA. The reverse primer was designed so that only 4-5 bases of the crucial 3′ side are exon 9 sequences and do not bind to UMOD mRNA that retains exon 10.

TABLE 2
qRT-PCR primers

| SEQ ID NO | Primer Name | Sequence |
|---|---|---|
| 6 | Human AS-UMOD Forward | gtctacctgcactgtgaagtc |
| 7 | Human AS-UMOD Reverse | agactttcaggagccctttc |
| 8 | Mouse AS-UMOD Forward | gtgactctacgagtgaacagtg |
| 9 | Mouse AS-UMOD Reverse | cagatgctcaggagcccttg |

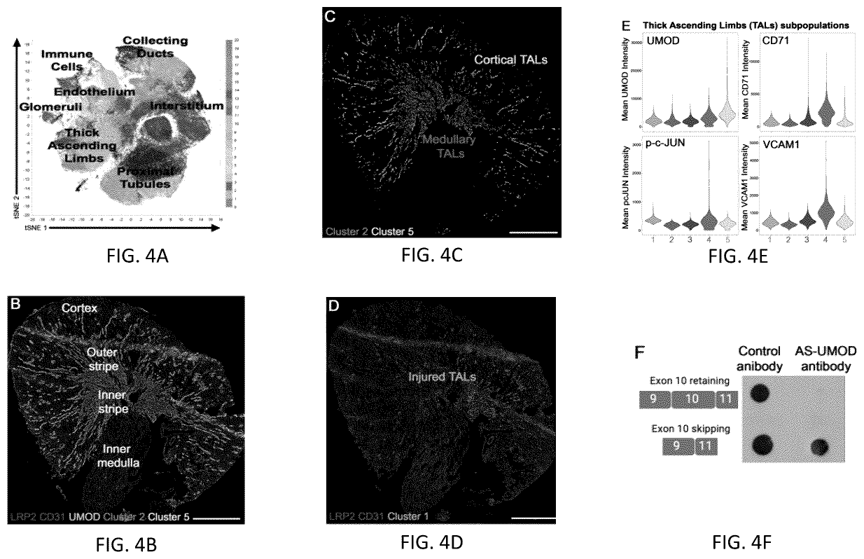

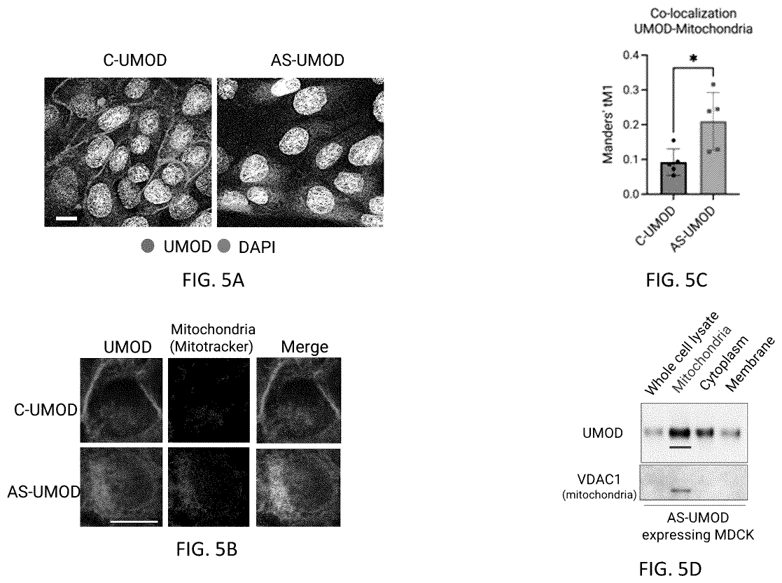

Spatial analysis in the kidney to detect RNA (e.g. single molecule fluorescence in situ hybridization (smFISH) spatial transcriptomics) (using technique described in Canela, V. H., et al., A spatially anchored transcriptomic atlas of the human kidney papilla identifies significant immune injury in patients with stone disease. Nat Commun, 2023. 14(1): p. 4140), and protein (highly multiplexed confocal and CODEX imaging) (using technique described in Ferreira, R. M., et al., Integration of spatial and single-cell transcriptomics localizes epithelial cell-immune cross-talk in kidney injury. JCI Insight, 2021. 6(12) and Winfree, S., et al., Integrated cytometry with machine learning applied to high-content imaging of human kidney tissue for <em>in-situ</em> cell classification and neighborhood analysis. bioRxiv, 2022: p. 2021.12.27.474025) were utilized to define the spatiotemporal dynamics of AS-UMOD during AKI. (FIGS. 4A-4F)

### Example 3: Development of SSOs

We have developed Splice-switching antisense oligonucleotides SSOs that can dynamically modulate alternative splicing of UMOD in vitro and in vivo. Based on in silico prediction, we designed, screened, and identified SSOs that can enhance endogenous AS-UMOD mRNA expression by 20-30 folds in MKTAL cells.

Four SSO sequences specific to mouse UMOD were created, as set forth in Table 3. When administered to mice, SSO Mm_UMOD 19/E10 (−13) (SEQ ID NO: 11) showed significant increase of AS-UMOD mRNA expression.

TABLE 3
Murine Splice-switching oligonucleotides (SSOs)

| SEQ ID NO | SSO name | SSO sequence |
|---|---|---|
| 10 | Mm_UMOD I9 (−36) | AAGACGAGAAACATGAGAAG |
| 11 | Mm_UMOD I9/E10 (−13) | TGGACACCTTTGTATGAAAC |
| 12 | Mm_UMOD E10 (2) | TGGACACTGAGGCCTGGACA |
| 13 | Mm_UMOD I10 (44) | GTACAGAAAGAACCTAAACTTA |

Some target sequences of SSOs effective for mice are 80-85% homologous to human sequence. Based on that, we also created three sequences for humans, as set forth in Table 4.

TABLE 4
Human Splice-switching antisense oligonucleotides (SSOs)

| SEQ ID NO | SSO name | SSO sequence |
|---|---|---|
| 14 | H_UMOD I9/E10 (−14) | tggacacctttggaggaaaac |
| 15 | H_UMOD E10 (3) | cttgagactgtggcctggac |
| 16 | H_UMOD I10 (41) | ctgcagaaaggacctgaactta |

### Example 4: AS-UMOD Induction Using SSOs Mitigates the Severity of Hypoxic Injury on Tal Cells In Vitro

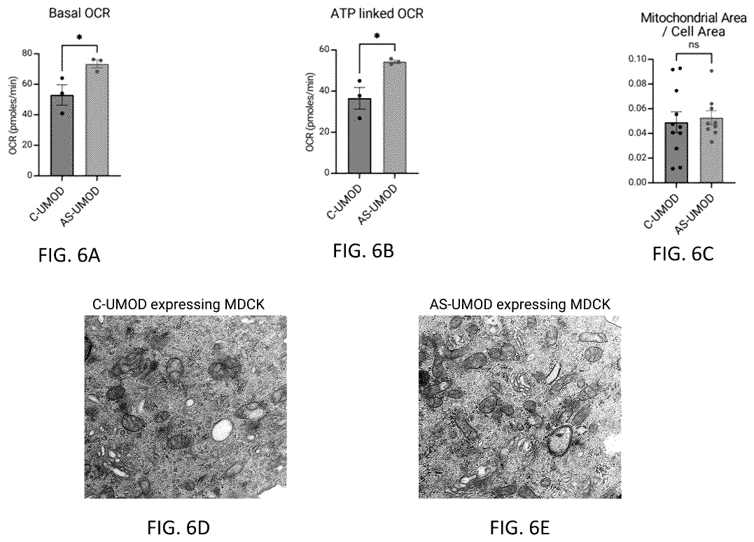

As described above, our data showed that AS-UMOD is up-regulated during AKI (FIGS. 3A). Additionally, our data shows the AS-UMOD is targeted to the mitochondria (FIGS. 5A-5D) and enhances mitochondrial function (FIG. 6A-6E). These changes and properties translate into cytoprotection and a favorable metabolism of TAL cells during injury. The existing evidence supports our position that TAL cell integrity is key for recovery from AKI.

Madin-Darby canine kidney (MDCK) cells and MKTAL are two versatile cell lines crucial for functional study of splice variants. MDCK cells express exogenous C-UMOD or AS-UMOD. MDCK cells are a well-established model for UMOD biology because there is no interference from endogenous UMOD in these cells. In contrast, MKTAL cells express endogenous UMOD, and retain properties of TAL cells.

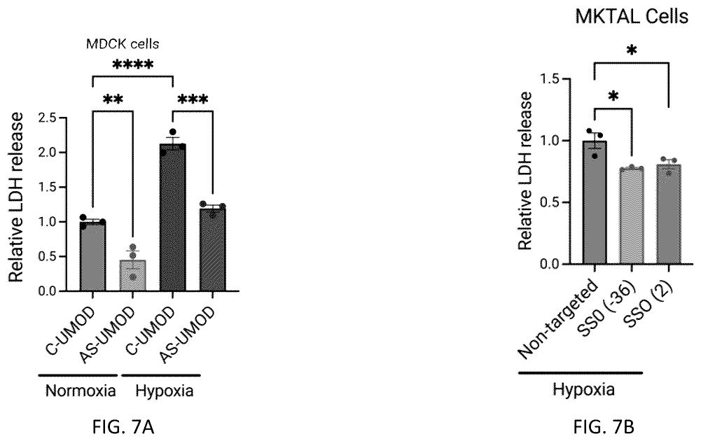

We have identified SSOs that enhance AS-UMOD expression in MKTAL cells, as we discussed above. Additional preliminary data suggests that AS-UMOD induction is protective against hypoxia in vitro both in MDCK and MKTAL cells (FIGS. 7A and 7B). These studies establish AS-UMOD-inducing SSO as a potential therapeutic strategy for AKI in subjects and establish a role for UMOD splicing in the protective metabolic changes during AKI.

We believe that AS-UMOD protects against hypoxia-induced injury of TAL cells by mitigating mitochondrial dysfunction-associated cell death. In our preliminary study, MDCK cells expressing AS-UMOD showed better cell viability in normoxia and hypoxia conditions compared to MDCK cells expressing C-UMOD (FIG. 7A). In addition, AS-UMOD induction using SSOs improved cell viability in MKTAL cells in hypoxia (FIG. 7B).

MDCK cells overexpressing UMOD have been a well-established model for UMOD biology. Various physiological functions of UMOD, such as intracellular targeting and trafficking, glycosylation, proteolytic cleavage, polymerization, and secretion have been demonstrated in MDCK cells. Another advantage of MDCK cells is that they do not express endogenous UMOD, enabling us to evaluate the precise function of UMOD variants when overexpressed. We established and utilized MDCK cells stably expressing C-UMOD or AS-UMOD using lentivirus.

MKTAL cells, an immortalized TAL cell line derived from mice, retain structural and functional properties of in vivo TAL cells and express endogenous UMOD. MKTAL cells are valuable because there are no commercially available TAL cell lines that express endogenous UMOD. Utilizing MKTAL cells is important to test the findings from MDCK cells, both within the endogenous UMOD and TAL cells contexts. SSOs enabled us to control AS-UMOD expression in MKTAL cells.

Mitochondrial dysfunction is linked to apoptosis, reactive oxygen species production (ROS) and activation of the cGAS-STING pathway though leaking of mitochondrial DNA. The latter two are also associated with various types of cell death and can also alter mitophagy. Therefore, it is very likely that the protective effect observed in FIG. 7A-B is due to the mitigation of a mitochondria-dysfunction associated cell death pathway.

MKTAL cells are transfected with non-targeted SSO (control) and AS-UMOD inducing SSOs (SEQ ID NOs 10-13) 48 hours after transfection, MTKAL cells are cultured in (1) normoxia condition (control), (2) hypoxia chamber for 6 hours (hypoxia) or (3) hypoxia chamber for 6 hours and subsequent normoxia condition for 16 hours (hypoxia+re-oxygenation). LDH release is measured to assess cell viability. TUNEL staining and Trypan-blue exclusion test are conducted to evaluate cell death. Intrinsic apoptosis pathways are evaluated by Cytochrome c, Bax and Bcl-2 protein expression. CellROX Deep Red Reagent is used to measure ROS levels. cGas/STING activation is measured by Western Blotting of phosphorylated and total STING protein expression (Chamma, H., et al., Protocol to induce and assess cGAS-STING pathway activation in vitro. STAR Protoc, 2022. 3(2): p. 101384). Additional characterization of cell death phenotype is performed by measuring the expression of cleaved caspases 1 and 3 (Guan, Y., et al., A single genetic locus controls both expression of DPEP1/CHMP1A and kidney disease development via ferroptosis. Nat Commun, 2021. 12(1): p. 5078), RIPK3, pMLKL (necroptosis), IL-1β, NLRP3 (pyroptosis) (Jiang, L., et al., hsa-miR-500a-3P alleviates kidney injury by targeting MLKL-mediated necroptosis in renal epithelial cells. FASEB J, 2019. 33(3): p. 3523-3535; von Massenhausen, A., et al., Phenytoin inhibits necroptosis. Cell Death Dis, 2018. 9(3): p. 359; Chen, H., et al., RIPK3-MLKL-mediated necroinflammation contributes to AKI progression to CKD. Cell Death Dis, 2018. 9(9): p. 878) and ACSL4 (ferroptosis) (Guan, Y., et al., A single genetic locus controls both expression of DPEP1/CHMP1A and kidney disease development via ferroptosis. Nat Commun, 2021. 12(1): p. 5078; Ide, S., et al., Ferroptotic stress promotes the accumulation of pro-inflammatory proximal tubular cells in maladaptive renal repair. Elife, 2021. 10) based on published reagents validated in our lab.

### Example 5: AS-UMOD Induction Using SSOs Mitigates AKI And Improves Its Course

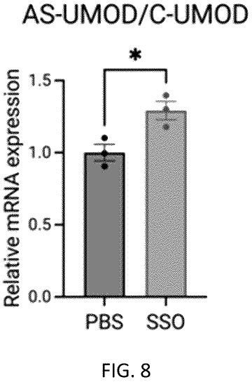

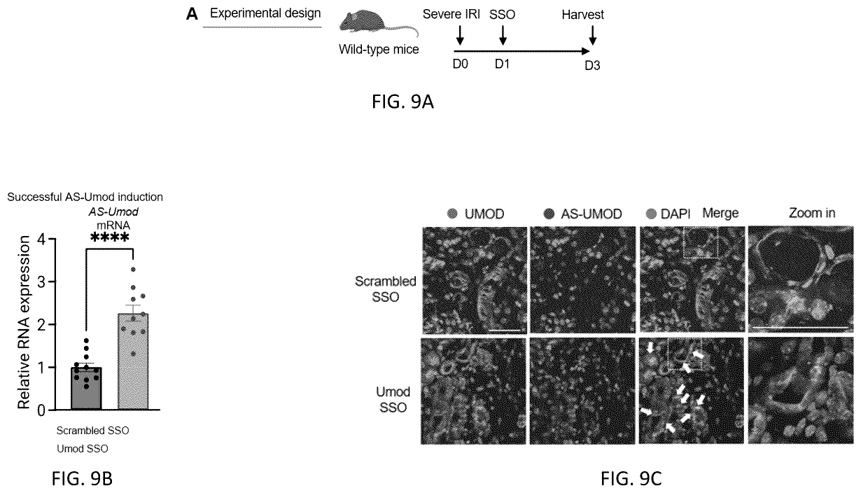

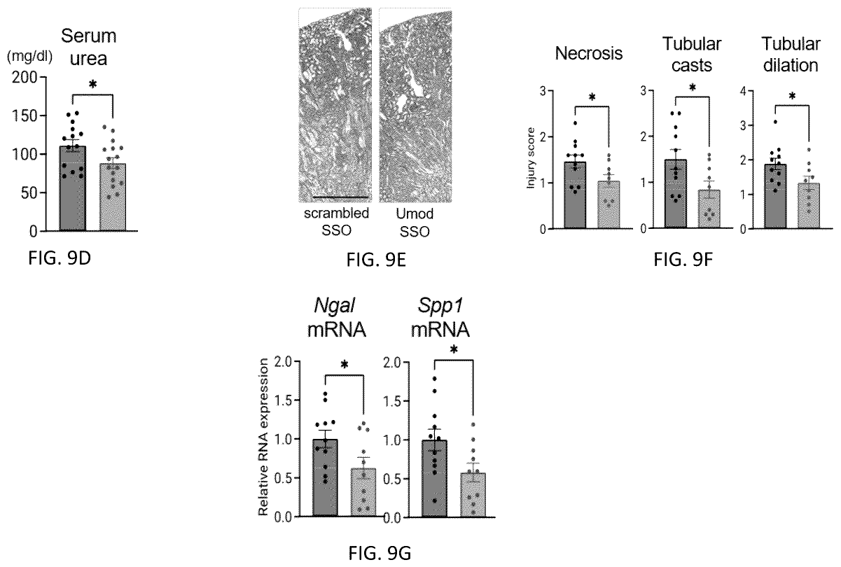

To evaluate AS-UMOD-inducing SSOs ability in vivo to reduce the severity of IRI and lead to faster repair by enhancing the protection of TAL segments, we inject AS-UMOD-inducing SSO (SEQ ID No. 11) to mice undergoing IRI surgery as shown in the experimental design of FIG. 9A, to establish their efficacy in reducing the severity and improving the course of injury. SSOs were administered 72 hours before harvesting the kidneys to quantify injury. Inducing AS-UMOD before AKI is inspired by the observation that preconditioning is effective for IRI (Bruzzese, L., et al., Hypoxic preconditioning in renal ischaemia-reperfusion injury: a review in pre-clinical models. Clin Sci (Lond), 2021. 135(23): p. 2607-2618). The feasibility, efficacy and dosing regimen are supported by our preliminary data showing that SSO administration enhanced AS-UMOD expression in vivo (FIG. 8).

Preliminary observations also did not reveal any evidence of toxicity, therefore, a higher dose of SSO is included to establish the dose response.

SSOs are labeled with 6-carboxyfluorescein (6-FAM), a fluorescent dye. Global kidney injury and the effect on TAL cells are analyzed using spatial analysis. As shown in FIG. 9B, the relative mRNA expression of AS-Umod and C-Umod normalized to Gapdh. The immunofluorescence images (FIG. 9C) were taken of the murine kidneys. White arrows indicate AS-UMOD which is induced in the cytosol of TAL cells after Umod SSO treatment. Scale Bar=50 μm. Graphs of the serum urea concentration are shown in FIG. 9D.

Based on the PAS-stained kidney sections (FIG. 9E), injury to the kidneys were quantified, as shown in FIG. 9F. Scale Bar=500 μm. The relative mRNA expression of injury-related genes is shown in FIG. 9G, normalized to Gapdh. Values are presented as mean±SEM. *p<0.05, ****p<0.0001. The administration (in vivo) of Umod SSO (25 mg/kg) after severe IRI successfully induced AS-UMOD at mRNA and protein levels and improved the course of AKI.

Recent progress in nucleic acid chemistry and pharmacology has led to many clinically approved SSOs or SSOs in phase 2-3 clinical trials, making SSOs one of the most rapidly evolving therapeutic strategies and the present invention an important advancement in the treatment of AKI and chronic kidney disease.

### EQUIVALENTS AND SCOPE

Those skilled in the art will recognize or be able to ascertain using no more than routine experimentation many equivalents to the specific embodiments described herein. The scope of the present invention is not intended to be limited to the above, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, it is to be understood that the invention encompasses all variations, combinations, and permutations in which one or more limitations, elements, clauses and descriptive terms, from one or more of the listed claims is introduced into another claim. For example, any claim that is dependent on another claim can be modified to include one or more limitations found in any other claim that is dependent on the same base claim.

Where elements are presented as lists, e.g., in Markush group format, it is to be understood that each subgroup of the elements is also disclosed, and any element(s) can be removed from the group. It should be understood that, in general, where the invention, or aspects of the invention is/are referred to as comprising particular elements, features, etc., certain embodiments of the invention or aspects of the invention consist, or consist essentially of, such elements, features, etc. For purposes of simplicity, those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranged can assume any specific value or sub- range within the stated ranges in different embodiments of the invention, to the tenth of the unit of the lower limit of the range, unless the context clearly dictates otherwise.

The term “about” or “approximately” means within an acceptable error range for the particular value as determined by one of the ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5% or up to 1% of a given value. Alternatively, the term can mean within an order of magnitude, for example within 5-fold, or within 2-fold, of a value. Where particular values are described in the application and claims, unless otherwise stated the term “about” meaning within an acceptable error range for the particular value should be assumed.

In addition, it is to be understood that any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the method of the invention can be excluded from any one or more claims, for any reason, whether or not related to the existence of prior art.

Each of the foregoing patents, patent applications and references is hereby incorporated by reference, particularly for the teaching referenced herein.

I claim:

1. A method of inducing exon-skipping of a AS-UMOD m-RNA in a TAL cell, the method comprising delivering to the cell a splice-switching antisense oligonucleotides (SSO) comprising a sequence selected from the group consisting of SEQ ID NOs: 14, 15, and 16, wherein the antisense oligonucleotide is up to 30 nucleotides in length and is capable of inducing exon-skipping of the UMOD pre-mRNA.

2. The method of claim 1 wherein the SSO sequence comprises SEQ ID NO: 14.

3. The method of claim 1 wherein the SSO sequence comprises SEQ ID NO: 15.

4. The method of claim 1 wherein the SSO sequences comprises SEQ ID NO: 16.

5. A method for enhancing endogenous alternatively spliced uromodulin (AS-UMOD) expression in a subject, comprising
administering splice-switching antisense oligonucleotides (SSO) comprising a sequence selected from the group consisting of SEQ ID NOs: 14, 15, and 16 to a subject.

6. The method of claim 5 wherein the enhanced endogenous AS-UMOD mRNA expression occurs in the kidney of the subject.

7. The method of claim 5 wherein the enhanced endogenous AS-UMOD mRNA expression occurs in the TAL cells of the subject.

8. The method of claim 5 wherein the subject is human.

9. The method of claim 5 wherein the AS-UMOD expressed lacks exon 10.

10. A method for treating acute kidney injury in a subject using the method of claim 5.

11. The method of claim 10 wherein the splice-switching antisense oligonucleotides (SSO) is administered to the subject less than 72 hours following acute kidney injury in the subject.

12. The method of claim 5 wherein the splice-switching antisense oligonucleotides (SSO) is administered to the subject prior to surgery on the kidney of the subject.

13. The method of claim 5 wherein the splice-switching antisense oligonucleotides (SSO) is administered to the subject less than 72 hours following acute kidney injury in the subject.

14. The method of claim 5 wherein the SSO is administered to the subject at a dosage of about 2 mg/kg.

15. A spice-switching antisense oligonucleotide (SSO) consisting of a sequence that has at least 90% identical over the entire length of SEQ ID NO: 14, 15 or 16.

16. Method of use of the composition contained the SSO of claim 12 in a subject suffering kidney injury.

17. A pharmaceutical composition comprising a pharmaceutically acceptable carrier and two or more antisense oligonucleotides complementary to AS-UMOD, wherein at least one of said antisense oligonucleotides thereof comprises a nucleic acid sequence as set forth in any one of SEQ ID Nos: 14, 15 or 16.
